(19) United States
(12) Patent Application Publication
(10) Pub. No.: US 2025/0243451 A1
(43) Pub. Date: Jul. 31, 2025

(54) RECOMBINANT ZYMOMONAS MOBILIS AND METHOD

(71) Applicant: Wuhan Ruijiakang Biotechnology Co., Ltd., Wuhan (CN)

(72) Inventors: Shihui YANG, Wuhan (CN); Weiwei BAO, Wuhan (CN); Qiqun PENG, Wuhan (CN)

(73) Assignee: Wuhan Ruijiakang Biotechnology Co., Ltd., Wuhan (CN)

(21) Appl. No.: 18/777,118

(22) Filed: Jul. 18, 2024

(30) Foreign Application Priority Data
Jan. 26, 2024 (CN) 2024101126994

(51) Int. Cl. C12N 1/20; C12N 15/74; C12P 7/42; C12R 1/01

(52) U.S. Cl. CPC C12N 1/205; C12N 15/74; C12P 7/42; C12R 2001/01

(57) ABSTRACT

Recombinant Zymomonas mobilis and method are provided. The recombinant Zymomonas mobilis carries a recombinant genome by genetic engineering means from the wild genome of ZM4 strain, and produces D-lactic acid.

### CROSS-REFERENCE TO RELATED APPLICATIONS

This disclosure claims priority to Chinese Patent Application NO: 202410112699.4, filed with China Intellectual Property Office on Jan. 26, 2024, the entire contents of which are incorporated herein by reference.

### SEQUENCE LISTING

The sequence listing xml file submitted herewith, named “Sequence_Listing.xml”, created on Jul. 15, 2024, and having a file size of 66,109 bytes, is incorporated by reference herein.

### TECHNICAL FIELD

This disclosure relates to Zymomonas mobilis. Specifically, this disclosure relates to recombinant Zymomonas mobilis and method.

### BACKGROUND

The statements herein provide background information relevant to the present disclosure only and do not necessarily constitute prior art.

Zymomonas mobilis, a natural ethanol-producing and facultative anaerobic Gram-negative bacterium, has unique Entner-Doudoroff (ED) metabolic pathway and high sugar fermentation efficiency. Moreover, as an ideal industrial cell factory, Zymomonas mobilis has the characteristics such as, high ethanol production, low biomass production, strong ethanol tolerance, high osmotic pressure resistance, and no need for additional oxygen during the fermentation process. At present, the production of polyhydroxybutyrate (abb. PHB), 2,3 butanediol (abb. 2,3-BDO), isobutanol and lactic acid have been realized in Zymomonas mobilis. Moreover, benefit by its high tolerance to lignocellulose hydrolysate, the production of ethanol from cellulose has already been commercialized in Zymomonas mobilis. Meanwhile, the mechanisms related to the tolerance of inhibitors in the lignocellulosic hydrolysates of Zymomonas mobilis has also been maturely studied. Additionally, by means of synthetic biology and metabolic engineering, Zymomonas mobilis could be modified into chassis cells that produce different platform compounds from the lignocellulosic hydrolysates.

### SUMMARY

In one aspect, embodiments disclose a recombinant Zymomonas mobilis. The recombinant Zymomonas mobilis is prepared from a Zymomonas mobilis subsp. mobilis ZM4=ATCC 31821 strain (abb. ZM4 strain, could be purchased from ATCC) by one or more options of knocking out the ZMO1360 loci of the ZM4 strain, replacing the ZMO0038 loci of the ZM4 strain with a LmldhA gene, replacing the ZMO1650 loci of the ZM4 strain with the LmldhA gene, replacing the ZMO1759 loci of the ZM4 strain with the LmldhA gene, replacing the ZMO1650 loci of the ZM4 strain with a 2,3-BDO operon.

In one aspect, embodiments disclose a method for preparing a recombinant Zymomonas mobilis. The method includes:
providing a Zymomonas mobilis subsp. mobilis ZM4=ATCC 31821 strain (abb. ZM4 strain, could be purchased from ATCC);
preparing a first editing plasmid for replacing the ZMO1650 loci of the ZM4 strain with a 2,3-BDO operon;
preparing a second editing plasmid for knocking out the ZMO1360 loci of the ZM4 strain;
preparing a third editing plasmid for replacing the ZMO0038 loci of the ZM4 strain with a LmldhA gene;
preparing a fourth editing plasmid for replacing the ZMO1759 loci of the ZM4 strain with the LmldhA gene;
preparing a fifth editing plasmid for replacing the ZMO1650 loci of the ZM4 strain with the LmldhA gene;
transforming one or more of the first editing plasmid, the second editing plasmid, the third editing plasmid, the fourth editing plasmid, the fifth editing plasmid into the ZM4 strain to obtain the recombinant Zymomonas mobilis.

In another aspect, embodiments disclose a method for preparing D-lactic acid. The method includes: fermenting the recombinant Zymomonas mobilis said above or prepare by the method said above in a medium containing glucose, and harvesting D-lactic acid from the fermentation product.

In another aspect, embodiments disclose a method for preparing D-lactic acid. The method includes: fermenting the recombinant Zymomonas mobilis said above or prepare by the method said above in a medium containing non-food biomass, and harvesting D-lactic acid from the fermentation product.

In another aspect, embodiments disclose uses by the recombinant Zymomonas mobilis said above or prepare by the method said above. The uses are selected from the group of preparing D-lactic acid, chiral separating lactic acid, synthesising polylactic acid, and synthesising bio-plastics.

### BRIEF DESCRIPTION OF DRAWINGS

FIG. 1 shows a global flow diagram of preparing the recombinants (ZM4-BDO, ZM4Δpdc-BDO, ZM4Δpdc-BDO-21dh, ZM4Δpdc-31dh) from ZM4 strain according to embodiments.

FIG. 2 shows a schematic diagram of preparing the ZM4-BDO strain from the ZM4 strain according to embodiments. “p12r-1650-BDO” refers to the first editing plasmid.

FIG. 3 shows a schematic diagram of preparing the ZM4Δpdc-BDO strain from the ZM4-BDO strain according to embodiments. “p12r-1360-ko” refers to the second editing plasmid.

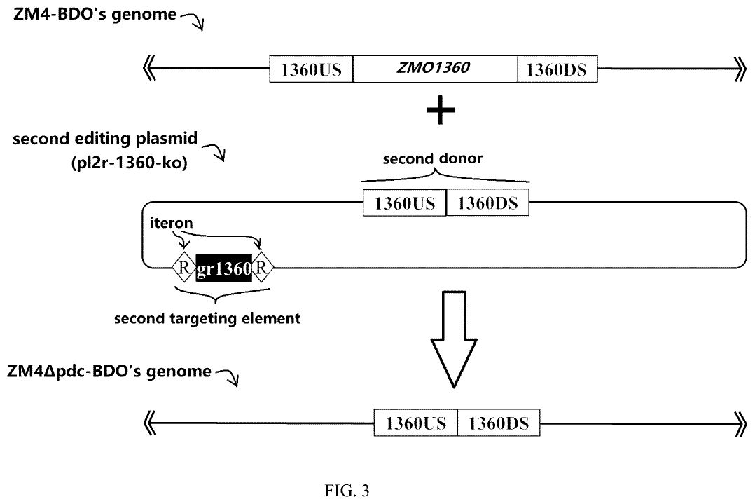

FIG. 4 shows a schematic diagram of preparing the ZM4Δpdc-BDO-21dh strain from the ZM4Δpdc-BDO strain according to embodiments. “p12r-0038-1dh” refers to the third editing plasmid, and “p12r-1759-1dh” refers to the fourth editing plasmid.

FIG. 5 shows a schematic diagram of preparing the ZM4Δpdc-31dh strain from the ZM4Δpdc-BDO-21dh strain according to embodiments. “p12r-1650Als-ldh” refers to the fifth editing plasmid.

FIG. 6 shows a glucose consumption curve of the ZM4Δpdc-31dh strain fermenting in mediums of RMG2, RMG5, RMG8, RMG10, RMG15 and CRH respectively according to embodiments.

FIG. 7 shows a D-lactic acid consumption curve of the ZM4Δpdc-31dh strain fermenting in mediums of RMG2, RMG5, RMG8, RMG10, RMG15 and CRH respectively according to embodiments.

FIG. 8 shows test results of C_{glu}, P_{LA}, Y_{LA}, fermentation cycle and η_{c} of the ZM4Δpdc-31dh strain fermenting in mediums of RMG2, RMG5, RMG8, RMG10, RMG15 and CRH respectively according to embodiments.

FIG. 9 shows test results of the fermenting products (Sample1, Sample1, and Sample3) of the ZM4Δpdc-31dh strain by HPLC according to embodiments.

### DETAILED DESCRIPTION OF THE EMBODIMENTS

In the description of this application, it is understood that the terms “first”, “second”, “third”, “fourth”, “fifth”, etc. are used only to distinguish the description and are not to be construed as indicating or implying relative order and importance.

Bio-plastics are materials having the same properties as petrochemical plastics manufactured from renewable resources such as food waste, lignocellulose waste, vegetable oil, corn and corn starch. In particular, compared with petrochemical plastics made from fossil fuels, the bio-plastics are biodegradable, renewable, less carbon footprint, more environmentally friendly, adaptable, and harmless to the environment. Polylactic acid is one of the most concerned bio-plastics in recent years. It has good biocompatibility, elasticity, thermoplastic properties, good molding ability and rigidity.

Polylactic acid is a non-toxic, environmentally friendly, widely used material for the human body and is “recognized as safe” (GRAS) by the U.S. Food and Drug Administration (FDA). Polylactic acid can be produced by renewable resources such as sugarcane, bagasse, corn, starch, food waste and the like. Lactic acid is used as a precursor of polylactic acid, and has two optical enantiomers, namely L-lactic acid and D-lactic acid. L-lactic acid has good bio-compatibility due to the characteristic of the L-rotation, can directly participate in human metabolism, and is widely applied in the fields of foods, medicines, cosmetics and the like. D-lactic acid is a synthesis precursor of polylactic acid and various chiral substances, and is widely used for chiral synthesis in the fields of medicine, pesticide, chemicals and the like, and also can be used for asymmetric synthesis of amino acid. Lactic acid is widely present in most microbial and animal metabolic pathways and can be produced by fermentation from renewable sources such as sugar cane, starch, corn and the like. Microbial fermentation is the most widely used method for industrial production of lactic acid. Due to its environmental friendliness, high conversion rate of sugar and acid, easy product separation and other advantages, it accounts for about 90% of the global production of lactic acid. In addition, the method of bio-synthesizing lactic acid is atomically economical due to its zero CO₂ emissions, contributing to the goal of global carbon neutrality.

Microorganisms used to produce lactic acid mainly include natural ones such as Lactic acidbacteria, Bacillus, Rhizopus, and engineered ones such as Saccharomyces cerevisiae. Different types of microorganisms (bacteria, fungi, yeasts, and algae) are able to produce lactic acid using different substrates. Lactic acid production strains can be divided into two categories: homologous fermentation strains and heterologous fermentation strains. Homologous fermentation strains are more suitable for industrial applications because of their higher lactic acid yield and easier downstream processing. However, the fermentation of heterologous fermentation strains is usually accompanied by the accumulation of ethanol, acetic acid and other by-products, which is limited by low lactic acid yield and high downstream processing cost.

For example, most lactic acid producing microorganisms will only be able to ferment sugars to produce L-lactic acid and not D-lactic acid. Si, H. et al produced L-lactic acid by fermenting corn stalk hydrolysate with Lactobacillus SP.L47. By controlling pH, the fermentation liquid containing 99.8 g/L L-lactic acid could be obtained, and the yield of L-lactic acid could reach 0.67 g/g (Si, H., liang, X., liu, S., xu, M., wang, J., and Hua, D. (2020) Efficient Production of L-Lactic Acid from Corn Straw Hydrolysate, IOP Conf.Ser. Earth environ. Sci.514, 052049. Doi: 10.1088/1755-1315/514/5/052049). Ahring, B. K et al. used Bacillus coagulans AD to produce L-lactic acid at a rate of 3.69 g/L/h, with a fermentation concentration of 35.2 g/L and a yield of 0.95 g/g (Ahring, B. K., Traverso, J. J., Murali, N., and Srinivas, K. (2016). Continuous Fermentation of Csugrified Corn stover Hydrolysate for the Production of Lactic Acid at High Yield and Productivity.Biochem.Eng.J.109, 162˜169. Doi: 10.1016/j.bej. 2016.01.012). Wei, C. et al. used Pediococcus acidilactici ZY271 to produce L-lactic acid at a rate of 1.81 g/L/h, with a fermentation liquid concentration of 35.2 g/L (Wei, C., liu, G., zhang, J., and Bao, 2J. (2018). Elevating Fermentation Yield of Cellulosic Lactic Acid in Calcium Lactate Form from Corn stover feedstock. Ind. Crops Prod.126, 415-420. Doi: 10.1016/J. Indcrop. 2018.10.041). Brock, S et al. used Sporolactobacillus inulinus DSM 20,348 to ferment zein acid hydrolysate with batch feeding to produce D-lactic acid with fermentation liquid content of 81 g/L and production rate of 3.85 g/L/h (Brock, S., kuenz, A., and Pruβe, U. (2019). Impact of Hydrolysis Methods on the Utilization of Agricultural Residues as Nutrient Source for D-Lactic Acid Production by Sporolactobacillus Inulinus. Fermantation 5 (12), 5010012. Doi: 10.3390/reference 5010012).

CN115851569A discloses a recombinant Z. mobilis for co-producing lactic acid and ethanol by non-grain biomass, its construction method and use. This recombinant Z. mobilis is an engineered strain of ZM4. This recombinant Z. mobilis has been constructed by introducing an exogenous lactate dehydrogenase gene with using the endogenous CRISPR-Cas gene editing system of Z. mobilis, increasing the copies of the exogenous lactate dehydrogenase gene, and changing the strength of its promoter to reduce the expression intensity of the ethanol metabolic pathway gene pdc. The recombinant Z. mobilis enhances lactic acid production capacity while also enabling the conversion of almost all biomass to lactic acid and ethanol products using non-grain biomass.

In one aspect, embodiments disclose a recombinant Zymomonas mobilis. The recombinant Zymomonas mobilis is prepared from a Zymomonas mobilis subsp. mobilis ZM4=ATCC 31821 strain (abb. ZM4 strain, could be purchased from ATCC) by one or more options of knocking out the ZMO1360 loci of the ZM4 strain, replacing the ZMO0038 loci of the ZM4 strain with a LmldhA gene, replacing the ZMO1650 loci of the ZM4 strain with the LmldhA gene, replacing the ZMO1759 loci of the ZM4 strain with the LmldhA gene, replacing the ZMO1650 loci of the ZM4 strain with a 2,3-BDO operon. Therein, the 2,3-BDO operon includes a Ptet promoter, a Als gene, a Pgap promoter, a Aldc gene and a Bdh gene. The 2,3-BDO operon has the nucleotide sequence of SEQ ID NO:1.

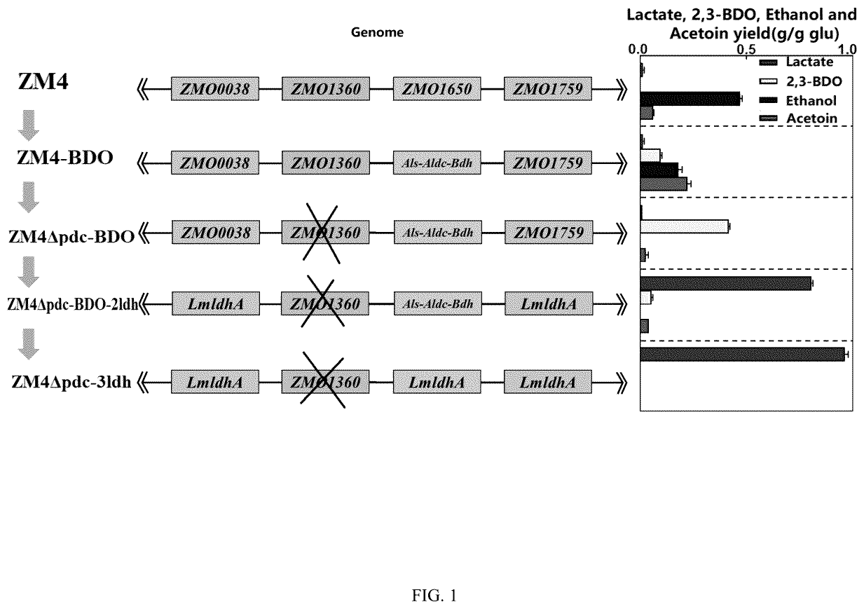

As shown in FIG. 1, through gene edit of ZMO1360 loci (gene complement (1375765 . . . 1377471), gene=“pdc”, under accession number CP023715.1 deposited in GenBank), ZMO0038 loci (gene complement (39623 . . . 40225) under accession number CP023715.1 deposited in GenBank), ZMO1650 loci (gene complement (1697900 . . . 1699036) under accession number CP023715.1 deposited in GenBank) and ZMO1759 loci (gene complement (1805297 . . . 1806253) under accession number CP023715.1 deposited in GenBank) in the wild genome (accession number CP023715.1 deposited in GenBank) of ZM4 strain, the lactate dehydrogenase gene LmldhA or 2,3-BDO operon are introduced to achieve genetic engineering transformation of ZM4 strain. Thus, the recombinant Zymomonas mobilis obtained is capable of synthesizing D-lactic acid and with the characteristics of high yield and high production efficiency.

In some embodiments, the recombinant Zymomonas mobilis is prepared from the ZM4 strain by knocking out the ZMO1360 loci of the wild genome, replacing the ZMO0038 loci of the wild genome with a LmldhA gene, replacing the ZMO1759 loci of the wild genome with the LmldhA gene, and replacing the ZMO1650 loci of the wild genome with a 2,3-BDO operon.

In some embodiments, the recombinant Zymomonas mobilis is prepared from the ZM4 strain by knocking out the ZMO1360 loci of the wild genome, replacing the ZMO0038 loci of the wild genome with a LmldhA gene, replacing the ZMO1650 loci of the wild genome with the LmldhA gene, and replacing the ZMO1759 loci of the wild genome with the LmldhA gene.

In one aspect, embodiments disclose a method for preparing a recombinant Zymomonas mobilis. The method includes:
providing a Zymomonas mobilis subsp. mobilis ZM4=ATCC 31821 strain (abb. ZM4 strain, could be purchased from ATCC);
preparing a first editing plasmid for replacing the ZMO1650 loci of the ZM4 strain with a 2,3-BDO operon, the 2,3-BDO operon includes a Ptet promoter, a Als gene, a Pgap promoter, a Aldc gene and a Bdh gene, the 2,3-BDO operon has the nucleotide sequence of SEQ ID NO:1;
preparing a second editing plasmid for knocking out the ZMO1360 loci of the ZM4 strain;
preparing a third editing plasmid for replacing the ZMO0038 loci of the ZM4 strain with a LmldhA gene;
preparing a fourth editing plasmid for replacing the ZMO1759 loci of the ZM4 strain with the LmldhA gene;
preparing a fifth editing plasmid for replacing the ZMO1650 loci of the ZM4 strain with the LmldhA gene;
transforming one or more of the first editing plasmid, the second editing plasmid, the third editing plasmid, the fourth editing plasmid, and the fifth editing plasmid into the ZM4 strain to obtain the recombinant Zymomonas mobilis.

In some embodiments, the recombinant Zymomonas mobilis is prepared by transforming the second editing plasmid, the third editing plasmid, and the fifth editing plasmid into the ZM4 strain.

In some embodiments, the recombinant Zymomonas mobilis is prepared by transforming the first editing plasmid, the second editing plasmid, the third editing plasmid, the fourth editing plasmid, and the fifth editing plasmid into the ZM4 strain.

In some embodiments, the first editing plasmid carries a first donor including the nucleotide sequence of SEQ ID NO:12 and a first targeting element. The first donor includes an upstream sequence of ZMO1650, the 2,3-BDO operon including the nucleotide sequence of SEQ ID NO:1 and a downstream sequence of ZMO1650. The first targeting element includes two iterons including the nucleotide sequence of SEQ ID NO:2 and a first guide RNA including the nucleotide sequence of SEQ ID NO:3 between the two iterons.

In some embodiments, the second editing plasmid carries a second donor including the nucleotide sequence of SEQ ID NO:26 and a second targeting element. The second donor includes an upstream sequence of ZMO1360 and a downstream sequence of ZMO1360. The second targeting element includes two iterons including the nucleotide sequence of SEQ ID NO:2 and a second guide RNA including the nucleotide sequence of SEQ ID NO:19 between the two iterons.

In some embodiments, the third editing plasmid carries a third donor including the nucleotide sequence of SEQ ID NO:37 and a third targeting element. The third donor includes an upstream sequence of ZMO0038, a LmldhA gene and a downstream sequence of ZMO0038. The third targeting element includes two iterons including the nucleotide sequence of SEQ ID NO:2 and a third guide RNA including the nucleotide sequence of SEQ ID NO:28 between the two iterons.

In some embodiments, the fourth editing plasmid carries a fourth donor including the nucleotide sequence of SEQ ID NO:45 and a fourth targeting element. The fourth donor includes an upstream sequence of ZMO1759, a LmldhA gene and a downstream sequence of ZMO1759. The fourth targeting element includes two iterons including the nucleotide sequence of SEQ ID NO: 2 and a fourth guide RNA including the nucleotide sequence of SEQ ID NO:38 between the two iterons.

In some embodiments, the fifth editing plasmid carries a fifth donor including the nucleotide sequence of SEQ ID NO:49 and a fifth targeting element. The fifth donor includes an upstream sequence of ZMO1650, a LmldhA gene and a downstream sequence of ZMO1650. The fifth targeting element includes two iterons including the nucleotide sequence of SEQ ID NO:2 and a fifth guide RNA including the nucleotide sequence of SEQ ID NO:46 between the two iterons.

In another aspect, embodiments disclose a method for preparing D-lactic acid. The method includes: fermenting the recombinant Zymomonas mobilis said above or prepare by the method said above in a medium containing glucose, and harvesting D-lactic acid from the fermentation product.

In another aspect, embodiments disclose a method for preparing D-lactic acid. The method includes: fermenting the recombinant Zymomonas mobilis said above or prepare by the method said above in a medium containing non-food biomass, and harvesting D-lactic acid from the fermentation product.

In another aspect, embodiments disclose uses by the recombinant Zymomonas mobilis said above or prepare by the method said above. The uses are selected from the group of preparing D-lactic acid, chiral separating lactic acid, synthesising polylactic acid, and synthesising bio-plastics.

### Prepare ZM4-BDO Strain

In some embodiments, the ZM4-BDO strain could be prepared by introducing a 2,3-BDO operon into the ZM4 strain.

Therein, the 2,3-BDO operon includes a Ptet promoter, a Als gene, a Pgap promoter, a Aldc gene and a Bdh gene. The 2,3-BDO operon has the nucleotide sequence of SEQ ID NO:1. The Als gene encodes an acetolactate synthase from Bacillus subtilis. The Aldc gene encodes an acetyl-lactate decarboxylase derived from Enterobacter cloacae. The Bdh gene encodes a 2,3-BDO dehydrogenase derived from Enterobacter cloacae. The Ptet promoter is a tetracycline-inducible promoter, and the Pgap promoter is an endogenous constitutive promoter in Zymomonas mobilis.

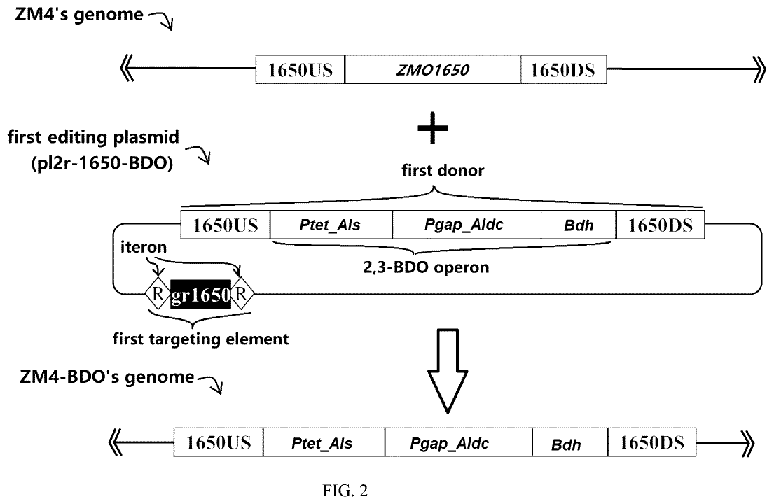

As shown in FIG. 2, the ZM4-BDO strain could be constructed by preparing the first editing plasmid (named p12r-1650-BDO) and transforming the p12r-1650-BDO plasmid into the ZM4 strain to introduce the 2,3-BDO operon to the ZMO1650 loci in the genome of the ZM4 strain. The p12r-1650-BDO plasmid carries a first donor including the nucleotide sequence of SEQ ID NO:12 and a first targeting element. The first donor includes an upstream sequence of ZMO1650, 2,3-BDO operon including the nucleotide sequence of SEQ ID NO:1 and a downstream sequence of ZMO1650. The first targeting element includes two first iterons including the nucleotide sequence of SEQ ID NO:2 and a first guide RNA (gr1650) including the nucleotide sequence of SEQ ID NO: 3 between the two iterons.

In some embodiments, the ZM4-BDO strain could be prepared by:

### 1. Prepare the First Guide RNA

The first guide RNA including the nucleotide sequence of SEQ ID NO:3 could be synthesized according to a 32 bp downstream sequence of the PAM site CCC loci from the ZMO1650 gene in the genome of ZM4 strain.

### 2. Prepare the First Targeting Plasmid

The first targeting plasmid that carries the first targeting element.

The first targeting plasmid could be synthesized by connecting the products of annealing primers (gr1650-F, SEQ ID NO:4; gr1650-R, SEQ ID NO:5) and a pEZ15Asp plasmid that contains a CRISPR-IF expression unit.

In some embodiments, the first targeting plasmid could be prepared by:
1) linearizing the pEZ15Asp plasmid by BsaI enzyme (Cat. NO:: HY-KE7007, MedChemExpress (MCE)) to obtain a linearized pEZ15Asp;
2) annealing primers (gr1650-F, SEQ ID NO:4; gr1650-R, SEQ ID NO:5); the annealing system could include 1 μLgr1650-F (10 μM), 1 μL gr1650-R (10 μM) and 8 μL ddH₂O;
3) ligating the annealing products with the linearized pEZ15Asp by T4 DNA Ligase (M0202V™, NEB) at 22° C. for 3˜6 h;
4) transforming the ligating products into E. coli DH5α (B528413-0010™, Sangon Biotech (Shanghai) Co., Ltd.);
5) screening positive colonies from the transformants of the step4) by spectinomycin plates;
6) verifying the positive colonies by Colony PCR and extracting the first targeting plasmid from the positive colonies.

Therein, the reaction system of ligation by T4 DNA Ligase included in 10 μL: 20˜40 ng linearized pEZ15Asp, 2 μL first guide RNA, 0.5 μL T4 DNA Ligase, 1 μL Buffer and surplus ddH₂O. The reaction procedure of Colony PCR included: pre-denaturation 98° C. for 3 min, 1 cycle; denaturation 98° C.10 s, anneal 55° C.10 s, extension 72° C., set 10 s/kb according to the fragment length, a total of 30 cycles; extension 72° C. for 2 min, 1 cycle; store at 12° C. for 2 min, 1 cycle.

The pEZ15Asp plasmid is a pEZ15A (https://www.molecularcloud.org/plasmid/TMpEZ15A/MC-0101148.html with Cat. NO: MC_0101148) with the spectinomycin resistance gene. pEZ15A plasmid could be prepared according to “Yang S, mohaghaeghi A, franden M A, et al, Metabolic engineering of Zymomonas mobilis for 2,3-butanediol production from lignocellulosic biomass sugars [J]. Biotechnol Biofuels, 2016, 9 (1): 189”. For obtaining pEZ15A with the different resistance genes, reference could be made to “Construction and Application of Plasmid pUC19-CM-D [J]. Agricultural Science & Technology, 2010, 11 (5): 31˜33”.

3. Prepare the First Editing Plasmid (p12r-1650-BDO)

In some embodiments, the first editing plasmid could be prepared by:
1) amplifying the 2,3-BDO operon (SEQ ID NO:1) with primers Als-F (SEQ ID NO:6) and Bdh-R (SEQ ID NO:7); the 2,3-BDO operon includes a Ptet promoter (1˜737 nt of SEQ ID NO: 1), a Als gene (738˜2449 nt of SEQ ID NO:1), a Pgap promoter (2450˜2762 nt of SEQ ID NO: 1), a Aldc gene (2763˜3542 nt of SEQ ID NO:1) and a Bdh gene (3543˜4332 nt of SEQ ID NO: 1);
2) amplifying the upstream sequence of ZMO1650 loci with primers 1650US-F (SEQ ID NO: 8) and 1650US-R (SEQ ID NO:9);
3) amplifying the downstream sequence of ZMO1650 loci with primers 1650DS-F (SEQ ID NO: 10) and 1650DS-R (SEQ ID NO:11);
4) orderly connecting the upstream sequence of ZMO1650 loci, the 2,3-BDO operon and the downstream sequence of ZMO1650 loci by Overlap PCR to get the first donor (SEQ ID NO:12);
5) reversely amplifying the first targeting plasmid with primers 15Afk-F (SEQ ID NO:13) and 15Afk-R (SEQ ID NO:14) to get the reverse sequence of the first targeting plasmid;
6) ligating the reverse sequence of the first targeting plasmid and the first donor with a mole ratio of 1:3 by Gibson Assembly;
7) transforming the product of ligation into E. coli DH5α;
8) screening the positive colonies by plates with containing spectinomycin from the transformants;
9) verifying the positive colonies by Colony PCR with primers pEZ15A-F (SEQ ID NO:15) and pEZ15A-R (SEQ ID NO:16);
10) extracting the first editing plasmid from the positive colonies.

### 4. Prepare the Competent ZM4 Strain

100 μL of frozen bacteria of ZM4 (Z. mobilis subsp.mobilis ZM4 ATCC 31821, ATCC) were removed from a −80° C. refrigerator, and inoculated into a frozen tube with containing 1 mL RM after thawing, and static cultured in a 30° C. incubator. The culture was shifted to a 50 mL blue cap bottle filled with 20 mL RM liquid medium after it grow to logarithmic stage (OD600 nm≈1.5˜2.0), and static cultured in a 30° C. incubator. After it grow to logarithmic stage (OD600 nm≈1.5˜2.0), the culture was shifted again to a 250 mL erlenmeyer flask filled with 200 mL RM liquid medium, and enabled the initial OD600 nm in a range of 0.025˜0.05, and static cultured at 30° C. and 100 rpm in a shaker. When the culture's OD600 nm was between 0.3 and 0.4, the thalli from the culture were collected with 4000 rpm centrifugation at normal temperature, then washed with sterile water for one time and 10% glycerol for two times, finally slowly re-suspended with 400 μL 10% glycerol, and sub-packaged 50 μL into a 1.5 mL EP tube.

### 5. Electro-Transform the First Editing Plasmid

1 μg of p12r-1650-dldh was added to a 1.5 mL EP tube with containing 50 μL of competent ZM4 strain, gently mixed and shifted into a 0.1 cm electroporation cuvette. And then the electroporation cuvette was placed into an electroporation instrument to electro-transform. Herein, the electro-transformation conditions were set as: 200Ω, capacitor: 25 μF, voltage: 1800 V. And 1 mL RM was added into the electroporation cuvette after electro-transforming, and static cultured in a 30° C. thermostatic incubator for 4˜6 h to get the transformants. And 200 μL solution of transformants were evenously coated on plates contained 100 μg/mL spectinomycin; sealed and anastrophic incubated in a 30° C. thermostatic incubator for 2˜3 days.

### 6. Mine for the ZM4-BDO Strain

After the growth of positive colonies on the plates contained 100 μg/mL spectinomycin, the positive colonies could be screened again by Colony PCR with primers 1650check-F (SEQ ID NO: 17) and 1650check-R (SEQ ID NO:18) and further verified by Sequencing. The confirmed positive colonies were the ZM4-BDO strain.

### Prepare the ZM4ΔPdc-BDO Strain

In some embodiments, the ZM4Δpdc-BDO strain could be prepared by introducing the 2,3-BDO operon into the ZM4 strain and knocking out a pdc gene from the ZM4 strain. The pdc gene (locates at the ZMO1360 loci of the genome of ZM4 strain) encodes a pyruvate decarboxylase involved in Ethanol biosynthetic pathway of ZM4 strain. The introduction of 2,3-BDO operon could shift the carbon metabolic flux into the production of 2,3-BDO.

As shown in FIG. 2, the ZM4Δpdc-BDO strain could be prepared by preparing the second editing plasmid and transforming the second editing plasmid into the ZM4-BDO strain. The second editing plasmid could knock out the ZMO1360 loci of ZM4-BDO strain or the ZM4 strain by its endogenous CRISPR-Cas gene editing system.

In some embodiments, the second editing plasmid (p12r-1360-ko) could be prepared by: providing a second targeting plasmid and inserting a second donor into the second targeting plasmid. Therein, the second targeting plasmid could be prepared by the way according to the preparation of the first targeting plasmid. The second targeting plasmid carries a second targeting element for guiding the ZMO1360 loci. The second targeting element includes two iterons including the nucleotide sequence of SEQ ID NO:2 and a second guide RNA (named gr1360) including the nucleotide sequence of SEQ ID NO:19 between the two iterons. The second targeting plasmid could be prepared by using the primers: gr1360-F (SEQ ID NO:20) and gr1360-R (SEQ ID NO: 21).

The second editing plasmid carries the second donor including the nucleotide sequence of SEQ ID NO:26 and a second targeting element. The second donor includes an upstream sequence of ZMO1360 and a downstream sequence of ZMO1360.

A step of inserting the second donor into the second targeting plasmid could include:
1) amplifying the upstream sequence of ZMO1360 loci with primers 1360US-F (SEQ ID NO: 22) and 1360US-R (SEQ ID NO:23);
2) amplifying the downstream sequence of ZMO1360 loci with primers 1360DS-F (SEQ ID NO: 24) and 1360DS-R (SEQ ID NO:25);
3) orderly connecting the upstream sequence of ZMO1360 loci and the downstream sequence of ZMO1360 loci by Overlap PCR to get the second donor (SEQ ID NO:26);
4) reversely amplifying the second targeting plasmid with primers 15Afk-F and 15Afk-R to get the reverse sequence of the second targeting plasmid;
5) ligating the reverse sequence of the second targeting plasmid and the second donor with a mole ratio of 1:3 by Gibson Assembly;
6) transforming the product of ligation into E. coli DH5α;
7) screening the positive colonies by plates with containing spectinomycin from the transformant;
8) verifying the positive colonies by Colony PCR with primers pEZ15A-F and pEZ15A-R; 9) extracting the second editing plasmid from the positive colonies.

### Prepare the ZM4ΔPdc-BDO-21dh Strain

In some embodiments, the ZM4Δpdc-BDO-21dh strain could be prepared by introducing a 2,3-BDO operon into the ZM4 strain, knocking out the pdc gene from the ZM4 strain, replacing the ZMO0038 loci and ZMO1759 loci with the LmldhA gene. The pdc gene (locates at the ZMO1360 loci of the genome of ZM4 strain) encodes a pyruvate decarboxylase involved in Ethanol biosynthetic pathway of ZM4 strain. The introduction of 2,3-BDO operon could shift the carbon metabolic flux into the production of 2,3-BDO. The replacements of ZMO0038 loci and ZMO1759 loci could introduce the heterologous lactic acid biosynthetic pathway.

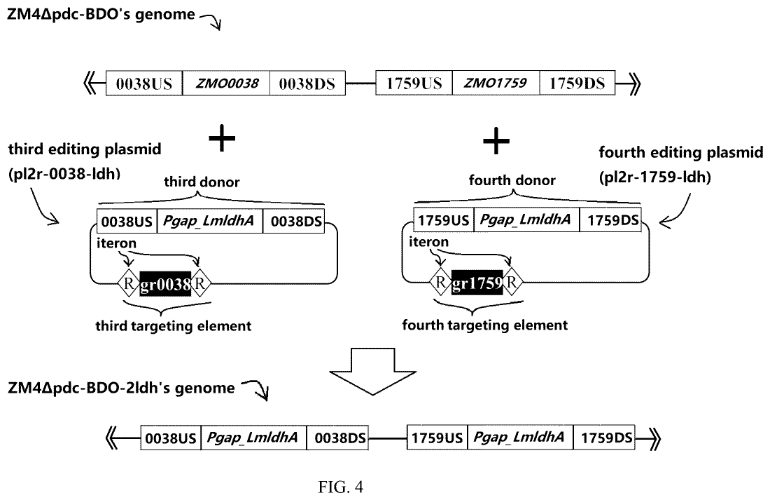

As shown in FIG. 4, the ZM4Δpdc-BDO-21dh strain could be prepared by providing the third editing plasmid (named p12r-0038-1dh) and the fourth editing plasmid (named p12r-1759-1dh), and transforming the third editing plasmid and the fourth editing plasmid into the ZM4Δpdc-BDO strain. Therein, the third editing plasmid could replace the ZMO0038 loci with the LmldhA gene by the endogenous CRISPR-Cas gene editing system of ZM4 strain. The fourth editing plasmid could replacing the ZMO1759 loci with the LmldhA gene by the endogenous CRISPR-Cas gene editing system of ZM4 strain.

1. Prepare the Third Editing Plasmid (Named p12r-0038-1dh)

In some embodiments, the third editing plasmid could be prepared by providing a third targeting plasmid and inserting a third donor into the third targeting plasmid. Therein, the third targeting plasmid could be prepared by the way according to the preparation of the first targeting plasmid. The third targeting plasmid carries a third targeting element for guiding the ZMO0038 loci. The third targeting element includes two iterons including the nucleotide sequence of SEQ ID NO: 2 and a third guide RNA (named gr0038) including the nucleotide sequence of SEQ ID NO: 27 between the two iterons. The third targeting plasmid could be prepared by using the primers: gr0038-F (SEQ ID NO: 28) and gr0038-R (SEQ ID NO:29).

A step of inserting the third donor into the third targeting plasmid could include:
1) amplifying the LmldhA gene (SEQ ID NO:30) with primers LmldhA-F (SEQ ID NO:31) and LmldhA-R (SEQ ID NO:32);
3) amplifying the upstream sequence of ZMO0038 loci with primers 0038US-F (SEQ ID NO: 33) and 0038US-R (SEQ ID NO:34);
4) amplifying the downstream sequence of ZMO0038 loci with primers 0038DS-F (SEQ ID NO: 35) and 0038DS-R (SEQ ID NO:36);
5) orderly connecting the upstream sequence of ZMO0038 loci, the LmldhA gene and the downstream sequence of ZMO0038 loci by Overlap PCR to get the third donor (SEQ ID NO:37);
6) reversely amplifying the third targeting plasmid with primers 15Afk-Fand 15Afk-R to get the reverse sequence of the third targeting plasmid;
7) ligating the reverse sequence of the third targeting plasmid and the third donor with a mole ratio of 1:3 by Gibson Assembly;
8) transforming the product of ligation into E. coli DH5α;
9) screening the positive colonies by plates with containing spectinomycin from the transformant;
10) verifying the positive colonies by Colony PCR with primers pEZ15A-F and pEZ15A-R;
11) extracting the third editing plasmid from the positive colonies.
   2. Prepare the Fourth Editing Plasmid (Named p12r-1759-1dh)

In some embodiments, the fourth editing plasmid could be prepared by providing a fourth targeting plasmid and inserting a fourth donor into the fourth targeting plasmid. Therein, the fourth targeting plasmid could be prepared by the way according to the preparation of the first targeting plasmid. The fourth targeting plasmid carries a fourth targeting element for guiding the ZMO1759 loci. The fourth targeting element includes two iterons including the nucleotide sequence of SEQ ID NO: 2 and a fourth guide RNA (named gr1759) including the nucleotide sequence of SEQ ID NO: 38 between the two iterons. The fourth targeting plasmid could be prepared by using the primers: gr1759-F (SEQ ID NO:39) and gr1759-R (SEQ ID NO:40).

A step of inserting the fourth donor into the fourth targeting plasmid could include:
1) amplifying the LmldhA gene (SEQ ID NO:30) with primers LmldhA-F (SEQ ID NO:30) and LmldhA-R (SEQ ID NO:31);
2) amplifying the upstream sequence of ZMO1759 loci with primers 1759US-F (SEQ ID NO: 41) and 1759US-R (SEQ ID NO:42);
3) amplifying the downstream sequence of ZMO1759 loci with primers 1759DS-F (SEQ ID NO: 43) and 1759DS-R (SEQ ID NO:44);
4) orderly connecting the upstream sequence of ZMO1759 loci, the LmldhA gene and the downstream sequence of ZMO1759 loci by Overlap PCR to get the fourth donor (SEQ ID NO:45);
5) reversely amplifying the fourth targeting plasmid with primers 15Afk-Fand 15Afk-R to get the reverse sequence of the fourth targeting plasmid;
6) ligating the reverse sequence of the fourth targeting plasmid and the fourth donor with a mole ratio of 1:3 by Gibson Assembly;
7) transforming the product of ligation into E. coli DH5α;
8) screening the positive colonies by plates with containing spectinomycin from the transformant;
9) verifying the positive colonies by Colony PCR with primers pEZ15A-F and pEZ15A-R;
10) extracting the fourth editing plasmid from the positive colonies.

### Prepare the ZM4ΔPdc-31dh Strain

In some embodiments, the ZM4Δpdc-31dh strain could be prepared by knocking out a pdc gene from the ZM4 strain, replacing the ZMO1650 loci, ZMO0038 loci and ZMO1759 loci with a LmldhA gene. The pdc gene (locates at the ZMO1360 loci of the genome of ZM4 strain) encodes a pyruvate decarboxylase involved in Ethanol biosynthetic pathway of ZM4 strain. The replacements of ZMO1650 loci, ZMO0038 loci and ZMO1759 loci could introduce the heterologous lactic acid biosynthetic pathway.

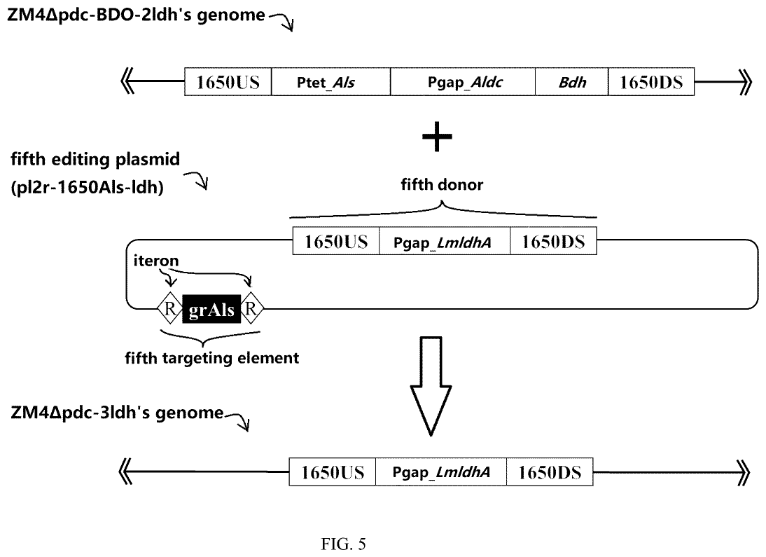

As shown in FIG. 5, the ZM4Δpdc-31dh strain could be prepared by providing the fifth editing plasmid (named p12r-1650-1dh) and transforming the fifth editing plasmid into the ZM4Δpdc-BDO-21dh strain. Therein, the fifth editing plasmid (named p12r-1650Als-ldh) could replacing the ZMO1650 loci with the LmldhA gene by the endogenous CRISPR-Cas gene editing system of ZM4 strain.

In some embodiments, the fifth editing plasmid could be prepared by providing a fifth targeting plasmid and inserting a fifth donor into the fifth targeting plasmid. Therein, the fifth targeting plasmid could be prepared by the way according to the preparation of the first targeting plasmid. The fifth targeting plasmid carries a fifth targeting element for guiding the ZMO1650 loci. The fifth targeting element includes two iterons including the nucleotide sequence of SEQ ID NO: 2 and a fifth guide RNA (named grA1s) including the nucleotide sequence of SEQ ID NO:46 between the two iterons. The fifth targeting plasmid could be prepared by using the primers: grA1s-F (SEQ ID NO:47) and grA1s-R (SEQ ID NO:48).

A step of inserting the fifth donor into the fifth targeting plasmid could include:
1) amplifying the LmldhA gene (SEQ ID NO:32) with primers LmldhA-F (SEQ ID NO:30) and LmldhA-R (SEQ ID NO:31);
2) amplifying the upstream sequence of ZMO1650 loci with primers 1650US-F (SEQ ID NO: 8) and 1650US-R (SEQ ID NO:9);
3) amplifying the downstream sequence of ZMO1650 loci with primers 1650DS-F (SEQ ID NO: 10) and 0038DS-R (SEQ ID NO:11);
4) orderly connecting the upstream sequence of ZMO1650 loci, the LmldhA gene and the downstream sequence of ZMO1650 loci by Overlap PCR to get the fifth donor (SEQ ID NO:49);
5) reversely amplifying the fifth targeting plasmid with primers 15Afk-Fand 15Afk-R to get the reverse sequence of the third targeting plasmid;
6) ligating the reverse sequence of the fifth targeting plasmid and the fifth donor with a mole ratio of 1:3 by Gibson Assembly;
7) transforming the product of ligation into E. coli DH5α;
8) screening the positive colonies by plates with containing spectinomycin from the transformant;
9) verifying the positive colonies by Colony PCR with primers pEZ15A-F and pEZ15A-R;

) extracting the fifth editing plasmid from the positive colonies.

### Tests

### 1. Fermentation

The fermentation for producing D-lactic acid by ZM4Δpdc-31dh strain was tested on different initial glucose concentration. The ZM4Δpdc-31dh strain was inoculated into a 50 mL conical flask with containing medium RMG2, RMG5, RMG8, RMG10, RMG15 or CRH (corncob hydrolysate) and with adding 100 g/L CaCO₃ respectively. The culture was fermented at 30° C., 100 rpm in a shaker. Samples at different time points in the fermentation were prepared by centrifuging (12000 rpm, 2 min) to get the supernatant, and filtered the supernatant by a 0.22 m filter for the HPLC test.

Therein, the RMG2 medium could be made by 20 g/L glucose, 10 g/L yeast extract and 2 g/L KH₂PO₄. The RMG5 medium could be made by 50 g/L glucose, 10 g/L yeast extract and 2 g/L KH₂PO₄. The RMG8 medium could be made by 80 g/L glucose, 10 g/L yeast extract and 2 g/L KH₂PO₄. The RMG10 medium could be made by 100 g/L glucose, 10 g/L yeast extract and 2 g/L KH₂PO₄. The RMG15 medium could be made by 150 g/L glucose, 10 g/L yeast extract and 2 g/L KH₂PO₄.

### 2. HPLC

Samples were tested on a HPLC (LC-20AD™, Shimadzu, Japan) system including an Aminex HPX-87H™ column (300 mm×7.8 mm, Bio-Rad) and a RID-20A differential refractometry detector. The concentrations of glucose, ethanol, lactic acid and 2,3-BDO in fermentation liquid were detected under the following conditions that included 0.005 mol/L H₂SO₄ for mobile phase, 0.5 mL/min for flow rate, 40° C. for detector temperature, 60° C. for column temperature, and 20 μL for injection volume.

The concentration of D-lactic acid was tested on a HPLC (1260 Infinity II, Agilent 1260™) system including a chiral column (Chirex 3126™ (D)-penicillamine) and a diode-array detector (1260 Infinity II HS™). The concentration of D-lactic acid was detected under the following conditions that included 0.002 mol/L CuSO₄ (the solvent was 5% isopropyl alcohol solution) for mobile phase, 1.0 mL/min for flow rate, 30° C. for diode-array detector temperature, 30° C. for column temperature, and 5 μL for injection volume.

The mobile phase could be prepared by dissolving CuSO₄·5H₂O into ddH₂O, and then adding isopropanol (chromatographic purity), vacuum ultra-filtering the liquid by 0.45 μm cellulose ester membrane, and ultrasonic degassing for 20˜30 min.

The concentrations of D-lactic acid and glucose in fermentation liquid were detected by these procedures.

The total consumption of sugar could be calculated by the formula: C_{sug}=g_{LA}/1; “P_{LA}” refers to the production of D-lactic acid, “C_{sug}” refers to the total consumption of sugar.

The yield of D-lactic acid could be calculated by the formula: Y_{LA}=g_{LA}/C_{glu}, “C_{glu}” refers to the total consumption of glucose, “Y_{LA}” refers to the yield of D-lactic acid.

The glucose conversion could be calculated by the formula: η_{c}=C_{sug}/C_{glu}.

### 3. Result

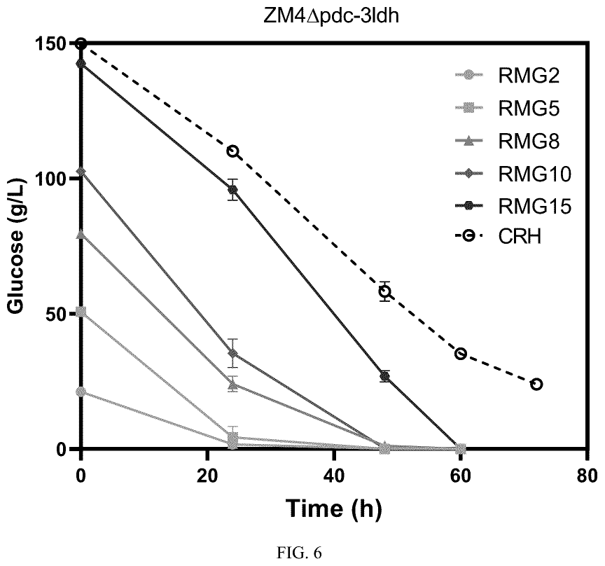

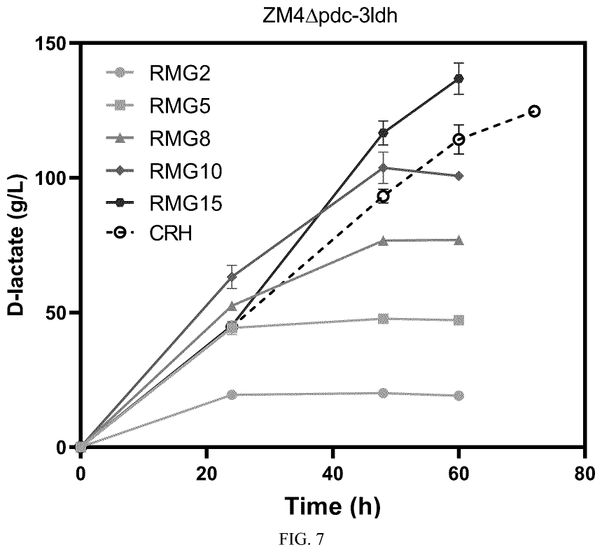

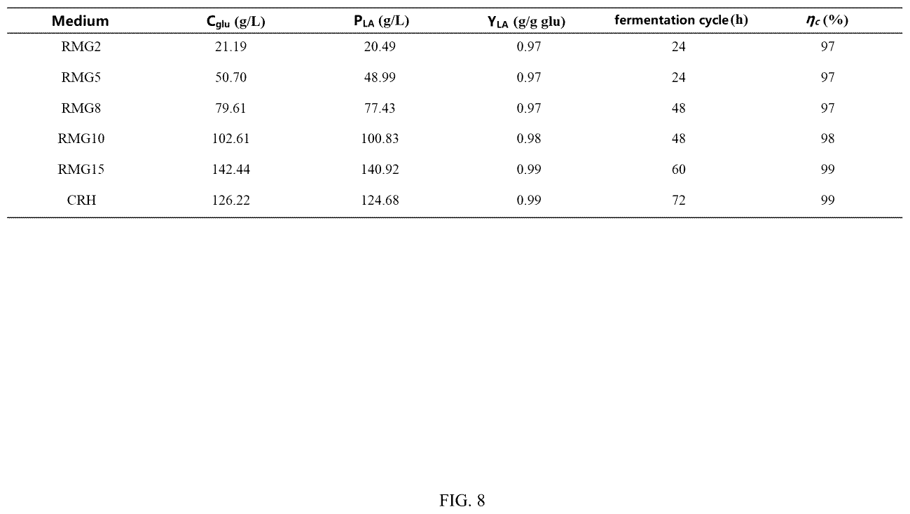

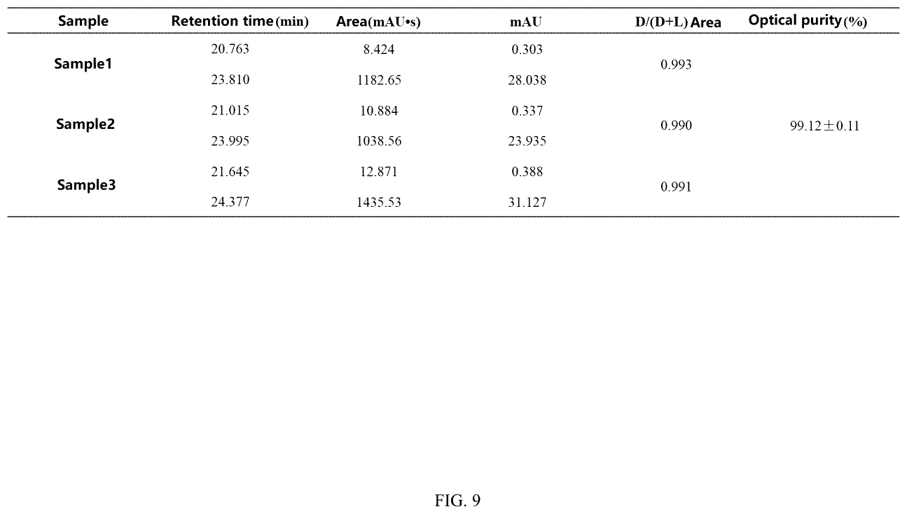

The consumption of glucose of ZM4Δpdc-31dh strain could be shown in FIG. 6. The production of D-lactic acid of ZM4Δpdc-31dh strain could be shown in FIG. 7. The conversion of glucose to lactic acid of ZM4Δpdc-31dh strain could be shown in FIG. 8. The optical purity of the producing lactic acid of ZM4Δpdc-31dh strain could be shown in FIG. 9. The ZM4Δpdc-31dh strain could consume almost all the glucose with an initial concentration of 20-150 g/L and convert it into D-lactic acid. The conversion rate of glucose to D-lactic acid is as high as 99%, and the optical purity of D-lactic acid is as high as 99.1%.

The above is only the preferred embodiments of this disclosure and is not intended to limit this disclosure. Any modification, equivalent replacement, improvement, etc., made within the spirit and principle of this disclosure shall be included in the scope of this disclosure.

The invention claimed is:

1-4. (canceled)

5. A engineered strain is a Zymomonas mobilis ZM4 strain that its ZMO1360 loci has been knocked out, its ZMO0038 loci and its ZMO1759 loci have been replaced with a LmldhA gene, and its ZMO1650 loci has been replaced with a 2,3-BDO expression element, said 2,3-BDO expression element is a connected sequence comprising a Ptet gene, a BsAls gene, a Pgap gene, a EcAldc gene and a EcBdh gene, said 2,3-BDO expression element has a nucleotide sequence shown as SEQ ID NO:1.

6. A engineered strain is a Zymomonas mobilis ZM4 strain that its ZMO1360 loci has been knocked out, its ZMO0038 loci, its ZMO1759 loci and its ZMO1650 loci have been replaced with a LmldhA gene.


SEQUENCE LISTING (49 sequences)

SEQ ID NO: 1 - DNA, 4332 bp, synthetic construct
  source 1..4332: mol_type = other DNA
gttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctggacatcattaattcctaatttttgttgacactctatcgttgatagagttattttaccactccctatcagtgatagagaaaagtattcaaatgatctaaagaggagaaaggatctcccatgacgaaagccacgaaagaacagaaatcgctggttaaaaatcggggtgctgaattagtcgttgattgcttagttgaacaaggcgttacccatgtttttggtatccccggtgcgaaaatcgatgcggtctttgatgctcttcaggataaaggtccggaaattatcgttgctcgccatgaacagaatgctgcctttatggcgcaagcagttggccgcttgacgggtaaacctggcgtcgttttggtcacgtctggccctggtgccagcaatcttgcgacgggtcttcttaccgctaataccgaaggtgatcctgttgtcgctcttgcgggtaatgtcattcgtgccgatcggttaaaacggacccatcaaagccttgataatgccgctttgtttcagcccatcaccaaatatagcgttgaagtccaggatgttaaaaatattcctgaagcagtcaccaatgccttccgtattgcgtcagctggccaggctggtgcagctttcgtcagctttcctcaagatgtcgtcaatgaagttacgaataccaaaaatgtccgtgctgttgcagccccgaaattaggccctgccgcagatgatgccatcagcgcagcgattgccaaaattcagacggccaaactgcccgttgtcttggttggtatgaaaggcggccgccccgaagcgattaaagccgtccgtaaacttctgaaaaaggtccagcttccgtttgtcgaaacgtatcaagccgcaggcacgttaagccgcgatcttgaagatcaatattttggtcgcatcggcttatttcgtaatcaacccggtgatttgttgcttgaacaggccgatgtcgttctgacgattggttatgatcctatcgaatatgatcctaaattttggaatattaatggcgatcgtacgatcatccatttagatgaaattatcgctgatatcgatcatgcatatcaacccgatcttgaacttattggtgatatcccgtccacgatcaatcatattgaacatgatgcggtcaaagttgaatttgctgaacgggaacaaaaaattctttcagatttgaaacaatatatgcatgaaggtgaacaagtccctgctgattggaaatccgatcgtgcgcatcctttagaaatcgttaaagaactgcgtaatgctgtcgatgatcatgtcaccgttacgtgtgatattggtagtcatgcaatctggatgtctcgttattttcgctcctatgaacctttgaccttgatgatttctaatggtatgcaaacccttggcgtcgcattgccttgggcaattggtgcgagcttggtcaaaccgggtgaaaaagttgtcagtgtctcaggcgatggtggcttcttgttttccgcgatggaattggaaacagctgttcgtcttaaagcacctatcgttcatatcgtctggaatgattccacctatgatatggttgcatttcaacagctgaaaaaatataatcgcacctctgcggttgattttggtaatattgatattgtcaaatatgccgaaagctttggtgcgaccggccttcgtgtcgaatctccggatcaacttgctgatgttttacgccagggtatgaatgccgaaggtccggtcattatcgatgttcctgttgattattctgataatattaatctggcgtcggataaactgcctaaagaatttggcgaactgatgaaaacgaaagctctgtacgcggccgcgttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaataaggtgttggccgcgatcgccggtaagtcggcacgttaaaaaatagctatggaatataatagctactaataagttaggagaataaacatgatgcattccagcgcatgcgattgcgaggcgtctttatgcgaaacattacgcggtttttcagctaaacacccagatagcgtcatctatcagacttctctgatgtctgccttgttgagtggagtttatgagggcgataccactattgcagatttattggcgcatggtgattttggcttgggcacgtttaatgaacttgacggagaaatgattgccttttcgagccaggtgtatcagttacgtgccgatggttccgcgcgtgctgccaaaccggaacagaaaaccccgtttgccgtcatgacgtggtttcaacctcaatatcgcaaaacgtttgatgcccccgtttctcgccagcaaattcatgatgttattgaccagcagattccctcggataatctgttttgtgctttgagaatcgacggcaactttcgtcatgctcatacccgtacggtgcctcgtcagacccctccctatcgcgccatgacagatgtcttagacgatcaacctgtgttccgtttcaatcagcgcgaaggagttttagttggttttcgcacacctcagcatatgcaaggcattaatgtcgccggttaccatgaacattttatcaccgatgatcgtcaaggtggtggtcacttactggactatcagttagaatctggtgtattaacttttggtgagattcacaagttgatgattgatttaccggcagattctgcgtttttgcaggcaaatttacacccttcgaacctggatgcagcaatacgttcagttgaaaattgagttgaagaggacgagcatgatgcagaaggtggcgctggtcactggttcgggtcaaggtataggtaaggctatcgctctgcggctggtgaaagacggctttgccgttgctattgctgattataatgacgaaaccgcccgggcagttgccgatgagattattcgcaatggcggcaacgcggttgccgtcaaagtcgatgtttcagaccgcgatcaggtcttcgctgccgttgaaaaagcccgcactgcattgggaggttttaacgtgattgtgaataacgcgggtattgccccttctacacccatagaatcgattacaccggaaatagtcgataaagtttataatattaatgtcaaaggcgttatttggggtatgcaagccgccattgatgcatttcgtaaagaaggccatggtggaaaaataattaatgcttgcagtcaggctggacatacgggtaatccggagttggccgtttattcgagctctaaattcgcagtgcggggactgacccagactgccgctagagatttagctccccttggcataacggttaatgcttattgtccggggatcgttaaaaccccgatgtgggcagaaatcgatcgccaggtttctgaagccgcaggcaaaccgttgggttatggcactgaaacctttgccaaacgtattacgcttggtcgcttgtctgaacccgaagatgtggctgcctgtgttagctatttggcgggtccggattctgattatatgaccggtcagtctctgttaattgacggtgggatggtattcaattaa

SEQ ID NO: 2 - DNA, 24 bp, synthetic construct
  source 1..24: mol_type = other DNA
gttcactgccgcacaggcagctta

SEQ ID NO: 3 - RNA, 32 nt, synthetic construct
  source 1..32: mol_type = other RNA
ccgtggtctcgcttaccaatacttcttttgaa

SEQ ID NO: 4 - DNA, 35 bp, synthetic construct
  source 1..35: mol_type = other DNA
gaaattcaaaagaagtattggtaagcgagaccacg

SEQ ID NO: 5 - DNA, 35 bp, synthetic construct
  source 1..35: mol_type = other DNA
gaacccgtggtctcgcttaccaatacttcttttga

SEQ ID NO: 6 - DNA, 47 bp, synthetic construct
  source 1..47: mol_type = other DNA
agaaagtaagcaatcgttaagacccactttcacatttaagttgtttt

SEQ ID NO: 7 - DNA, 46 bp, synthetic construct
  source 1..46: mol_type = other DNA
atcaagaccgcacctttaattgaataccatcccaccgtcaattaac

SEQ ID NO: 8 - DNA, 34 bp, synthetic construct
  source 1..34: mol_type = other DNA
accagctcaccgtctccgatccgccctatggtct

SEQ ID NO: 9 - DNA, 47 bp, synthetic construct
  source 1..47: mol_type = other DNA
ggttgttgatcgaacgattgcttactttcttctctggaatatcctcc

SEQ ID NO: 10 - DNA, 36 bp, synthetic construct
  source 1..36: mol_type = other DNA
gctgttgaatattaaggtgcggtcttgattagcctt

SEQ ID NO: 11 - DNA, 35 bp, synthetic construct
  source 1..35: mol_type = other DNA
agatctgatatcactgtgctatccgcttggctctc

SEQ ID NO: 12 - DNA, 5834 bp, synthetic construct
  source 1..5834: mol_type = other DNA
tcaccgatccgccctatggtctgaccaatcttgatgaatccgacatcaaagagatgaactacaatggtgtgttccgtctctctcccgatggtcgtttagatttgatcgaagctggtcttagccgcccgaatggtttggctctgtccccggatgaaacgaaactttatgtttctaactcagaccgcgccagcccgaatatctgggtttattctttggatagcaatggcttgccaaccagccggactttgctgcggaacttccgcaaggaatatttcgatcagggtctcgccggtcttcctgatggtatgaatatcgacaaacagggcaatctttttgcttctgctccgggcggtatctatatttttgcaccagatggcgaatgcttgggtcttatttctggtaacccaggtcagccgttgtcgaactgctgctttggtgaaaaaggccagaccttgttcatctcggcttctcacaatgttgtgcgcgttcgcaccaaaacctttggataattattgtctgtcataaaaatataaaaaacagataatatacaaaattcaggcccgttttaagggaagtaatagaagaggatgccaaaaggcaaattcctgacgggccctgataagagaaagaacaaaagaattctttcttaagaatactctataatttggctatctttcttttctgaccgctttattgtcggaggatattccagagaagaaagtaagcaatcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctggacatcattaattcctaatttttgttgacactctatcgttgatagagttattttaccactccctatcagtgatagagaaaagtattcaaatgatctaaagaggagaaaggatctcccatgacgaaagccacgaaagaacagaaatcgctggttaaaaatcggggtgctgaattagtcgttgattgcttagttgaacaaggcgttacccatgtttttggtatccccggtgcgaaaatcgatgcggtctttgatgctcttcaggataaaggtccggaaattatcgttgctcgccatgaacagaatgctgcctttatggcgcaagcagttggccgcttgacgggtaaacctggcgtcgttttggtcacgtctggccctggtgccagcaatcttgcgacgggtcttcttaccgctaataccgaaggtgatcctgttgtcgctcttgcgggtaatgtcattcgtgccgatcggttaaaacggacccatcaaagccttgataatgccgctttgtttcagcccatcaccaaatatagcgttgaagtccaggatgttaaaaatattcctgaagcagtcaccaatgccttccgtattgcgtcagctggccaggctggtgcagctttcgtcagctttcctcaagatgtcgtcaatgaagttacgaataccaaaaatgtccgtgctgttgcagccccgaaattaggccctgccgcagatgatgccatcagcgcagcgattgccaaaattcagacggccaaactgcccgttgtcttggttggtatgaaaggcggccgccccgaagcgattaaagccgtccgtaaacttctgaaaaaggtccagcttccgtttgtcgaaacgtatcaagccgcaggcacgttaagccgcgatcttgaagatcaatattttggtcgcatcggcttatttcgtaatcaacccggtgatttgttgcttgaacaggccgatgtcgttctgacgattggttatgatcctatcgaatatgatcctaaattttggaatattaatggcgatcgtacgatcatccatttagatgaaattatcgctgatatcgatcatgcatatcaacccgatcttgaacttattggtgatatcccgtccacgatcaatcatattgaacatgatgcggtcaaagttgaatttgctgaacgggaacaaaaaattctttcagatttgaaacaatatatgcatgaaggtgaacaagtccctgctgattggaaatccgatcgtgcgcatcctttagaaatcgttaaagaactgcgtaatgctgtcgatgatcatgtcaccgttacgtgtgatattggtagtcatgcaatctggatgtctcgttattttcgctcctatgaacctttgaccttgatgatttctaatggtatgcaaacccttggcgtcgcattgccttgggcaattggtgcgagcttggtcaaaccgggtgaaaaagttgtcagtgtctcaggcgatggtggcttcttgttttccgcgatggaattggaaacagctgttcgtcttaaagcacctatcgttcatatcgtctggaatgattccacctatgatatggttgcatttcaacagctgaaaaaatataatcgcacctctgcggttgattttggtaatattgatattgtcaaatatgccgaaagctttggtgcgaccggccttcgtgtcgaatctccggatcaacttgctgatgttttacgccagggtatgaatgccgaaggtccggtcattatcgatgttcctgttgattattctgataatattaatctggcgtcggataaactgcctaaagaatttggcgaactgatgaaaacgaaagctctgtacgcggccgcgttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaataaggtgttggccgcgatcgccggtaagtcggcacgttaaaaaatagctatggaatataatagctactaataagttaggagaataaacatgatgcattccagcgcatgcgattgcgaggcgtctttatgcgaaacattacgcggtttttcagctaaacacccagatagcgtcatctatcagacttctctgatgtctgccttgttgagtggagtttatgagggcgataccactattgcagatttattggcgcatggtgattttggcttgggcacgtttaatgaacttgacggagaaatgattgccttttcgagccaggtgtatcagttacgtgccgatggttccgcgcgtgctgccaaaccggaacagaaaaccccgtttgccgtcatgacgtggtttcaacctcaatatcgcaaaacgtttgatgcccccgtttctcgccagcaaattcatgatgttattgaccagcagattccctcggataatctgttttgtgctttgagaatcgacggcaactttcgtcatgctcatacccgtacggtgcctcgtcagacccctccctatcgcgccatgacagatgtcttagacgatcaacctgtgttccgtttcaatcagcgcgaaggagttttagttggttttcgcacacctcagcatatgcaaggcattaatgtcgccggttaccatgaacattttatcaccgatgatcgtcaaggtggtggtcacttactggactatcagttagaatctggtgtattaacttttggtgagattcacaagttgatgattgatttaccggcagattctgcgtttttgcaggcaaatttacacccttcgaacctggatgcagcaatacgttcagttgaaaattgagttgaagaggacgagcatgatgcagaaggtggcgctggtcactggttcgggtcaaggtataggtaaggctatcgctctgcggctggtgaaagacggctttgccgttgctattgctgattataatgacgaaaccgcccgggcagttgccgatgagattattcgcaatggcggcaacgcggttgccgtcaaagtcgatgtttcagaccgcgatcaggtcttcgctgccgttgaaaaagcccgcactgcattgggaggttttaacgtgattgtgaataacgcgggtattgccccttctacacccatagaatcgattacaccggaaatagtcgataaagtttataatattaatgtcaaaggcgttatttggggtatgcaagccgccattgatgcatttcgtaaagaaggccatggtggaaaaataattaatgcttgcagtcaggctggacatacgggtaatccggagttggccgtttattcgagctctaaattcgcagtgcggggactgacccagactgccgctagagatttagctccccttggcataacggttaatgcttattgtccggggatcgttaaaaccccgatgtgggcagaaatcgatcgccaggtttctgaagccgcaggcaaaccgttgggttatggcactgaaacctttgccaaacgtattacgcttggtcgcttgtctgaacccgaagatgtggctgcctgtgttagctatttggcgggtccggattctgattatatgaccggtcagtctctgttaattgacggtgggatggtattcaattaaaggtgcggtcttgattagccttgaagcctgacttcaaggctaagagaaaaaatagattttagataaagacaaccaaacatcctaagaataggatgttaatttataaccacgccgtgtttttttgcccataattcaagctggtcaccaatattgctggcaccgcttgcaaccaattcccgaataaagggcgcaaaagcatcccgatccaaagagcggatcattgccttgataggccctatcgccactggtgtgatagaaagccgttttaagcccaatccgatgagtgccatggccgtcagagtacgaccacccatttccccgcaaatgccgaccttacaacccgcagcattggcctgatcgataatccgtgacaagaaacgcagcaaagccggagagagccagtcatatctttctgccagcttgggataagcacgatcggctgcaaaaaagaactggatcagatcattagtgccaatcgacaagaaatcgagcttgggcaacagaatatcaagcgtctcggctaaggaaggcacctctagcatcgagccataataaattctcttcgcgcgcggtttgtcatgtttggcaagccaagcattttgcttttcaaagacagcgcgagcctgttcatattcccatggctcggcaatcatcgggaacatgacataaagcgtttttcccgcagccccttcaatcaatgcccgtgcctgaaccttgagcagggctttcttttcgagagccaagcggatagcacagtgatatcagatctcga

SEQ ID NO: 13 - DNA, 31 bp, synthetic construct
  source 1..31: mol_type = other DNA
agtgatatcagatctcgagctcggtacccgg

SEQ ID NO: 14 - DNA, 20 bp, synthetic construct
  source 1..20: mol_type = other DNA
agacggtgagctggtgacct

SEQ ID NO: 15 - DNA, 22 bp, synthetic construct
  source 1..22: mol_type = other DNA
ggcaaagccaccctatttttag

SEQ ID NO: 16 - DNA, 20 bp, synthetic construct
  source 1..20: mol_type = other DNA
cacttcactgacaccctcat

SEQ ID NO: 17 - DNA, 21 bp, synthetic construct
  source 1..21: mol_type = other DNA
ccgatggcaaaatctgggttg

SEQ ID NO: 18 - DNA, 20 bp, synthetic construct
  source 1..20: mol_type = other DNA
taccgagacgggaaagacag

SEQ ID NO: 19 - RNA, 32 nt, synthetic construct
  source 1..32: mol_type = other RNA
caatgagttatactgtcggtacctatttagcg

SEQ ID NO: 20 - DNA, 36 bp, synthetic construct
  source 1..36: mol_type = other DNA
gaaacaatgagttatactgtcggtacctatttagcg

SEQ ID NO: 21 - DNA, 36 bp, synthetic construct
  source 1..36: mol_type = other DNA
gaaccgctaaataggtaccgacagtataactcattg

SEQ ID NO: 22 - DNA, 40 bp, synthetic construct
  source 1..40: mol_type = other DNA
agatctgatatcacttacctgattacgacaaatcaagcag

SEQ ID NO: 23 - DNA, 43 bp, synthetic construct
  source 1..43: mol_type = other DNA
ctgattcctttttaaataaacttagagcttaaggcgaaaagcc

SEQ ID NO: 24 - DNA, 35 bp, synthetic construct
  source 1..35: mol_type = other DNA
ttaaaaaggaatcagaaccagatgtagaaacgtag

SEQ ID NO: 25 - DNA, 35 bp, synthetic construct
  source 1..35: mol_type = other DNA
accagctcaccgtctattgtaggcggctggattgt

SEQ ID NO: 26 - DNA, 1364 bp, synthetic construct
  source 1..1364: mol_type = other DNA
tacctgattacgacaaatcaagcagggctggaatggtttggaacagaaatagagggaatatgccatctttctaaagaagagcgatggcatccctttccctgacgtttaagcgagcgcagcgacgccttttgccaaacgagagaatttccgtgaagccgtgtttttatgcaaaacacctttagaaacgccacggaataattccggctgtgcgcttgctaaagcggtttcagcttcggatttgttaccagcagcaatagcagattcgacttttttaatgaaagtacgaatgcggttgacgcggttaccgttaatttcagcgcgacggtcgttacgacggatgcgcttttttgcctgcggcgtattcgccatgcttgtcctcgatgtaatcagaaaataaatagttaaggcactgacttcaataattcagcccttacgaagaagaagtctttatggcttttctttttatcggtcaagagccagccctgcttagcaagggattctgtaacaattttatagaaaagaaaaacaaagctgtcatttatgtttcaataagacatttttgttgagagggaaaatcaggaaaaaaaggaacgcgtaaaaaagcgctatgcctcctgccccatcatttgagtcgtcttggataaaaaaagcccgcccggtaaaaccggacgggcttttcgccttaagctctaagtttatttaaaaaggaatcagaaccagatgtagaaacgtagccgagacctaaagcagccgaagacgcctgttttaaaatttttccggctgttgtcatgaacggcctcatcaattccaaacaagacagggcaggatcaatccaaaactgcccgaggcgagcgaccggctttatcaatgagctgttacagatattataccgcaacactgacaaagcctttccctccgtctgagctatcaaagcggaaacgtttccgtaaccggtctgcccctctggtgatattttattgtcgccaagtcaacccaaagttgtgcataaacaaaatttgttaagcccaagccaacccttaaccacgccctaagagaggataaaaaagggcttcttcttttgaaggatagcccgtgaatcttgtggtcaaaaccgttgttttcagtatttatttttcgctttcgaaagaagatgaaagggatattctttggataaaaaaaggcggtcttgtcccgagtaaaaacagactcaatttttttcgattaaagttaatattttttcgcgacccaaaagagacgaagatccgatatcgtcgccgtcatcttgttacagactattgcaaagagtttggatagaaaagaaataaggaaccgtgtgactttatgacaatccagccgcctacaat

SEQ ID NO: 27 - RNA, 32 nt, synthetic construct
  source 1..32: mol_type = other RNA
gcgtccagcaaaatacgccttctattgatgaa

SEQ ID NO: 28 - DNA, 36 bp, synthetic construct
  source 1..36: mol_type = other DNA
gaaagcgtccagcaaaatacgccttctattgatgaa

SEQ ID NO: 29 - DNA, 36 bp, synthetic construct
  source 1..36: mol_type = other DNA
gaacttcatcaatagaaggcgtattttgctggacgc

SEQ ID NO: 30 - DNA, 1301 bp, synthetic construct
  source 1..1301: mol_type = other DNA
gttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatatagtagctacttaataagttaggagaataaacatgaagatttttgcttacggcattcgtgatgatgaaaagccatcacttgaagaatggaaagcggctaacccagagattgaagtggactacacacaagagctattgacacctgaaacagttaagttggctgagggatcagattcagctgttgtttaccaacaactggactatacacgtgaaacattgacagctttagctaacgttggtgttactaacttgtcattgcgtaacgttggtacagataacattgattttgatgcagcacgtgaatttaactttaacatttcaaatgttcctgtttattcaccaaatgctattgcagaacactcaatgattcaattatctcgtttgctacgtcgcacgaaagcattggatgccaaaattgctaagcacgacttgcgctgggcaccaacaattggacgtgaaatgcgtatgcaaacagttggtgttattggtacaggccatattggccgtgttgctattaacattttgaaaggctttggggcaaaggttattgcttatgataagtacccaaatgctgaattgcaagcagaaggtttgtacgttgacacattagacgaattatatgcacaagctgatgcaatttcattgtatgttcctggtgtgcctgaaaaccatcatctaatcaatgcagaggctattgctaagatgaaggatggcgtggttatcatgaatgctgcgcgtggtaatttgatggacattgatgctattattgatggtttgaattctggtaagatttcagacttcggtatggacgtttatgaaaatgaagttggcttgttcaatgaagattggtctggtaaagaattcccagatgctaagattgctgacttgatttcacgcgaaaatgtattggttacgccacatacggctttctatacaactaaagctgttctagaaatggttcaccaatcatttgatgcagcagttgctttcgccaaaggtgagaagccagctattgctgttgaatattaa

SEQ ID NO: 31 - DNA, 40 bp, synthetic construct
  source 1..40: mol_type = other DNA
ggcgtcgggcgtgagttcgatcaacaacccgaatcctatc

SEQ ID NO: 32 - DNA, 46 bp, synthetic construct
  source 1..46: mol_type = other DNA
ctgaatatttaacgattaatattcaacagcaatagctggcttctca

SEQ ID NO: 33 - DNA, 40 bp, synthetic construct
  source 1..40: mol_type = other DNA
agatctgatatcactcaacagatcaaccggcaatttatcc

SEQ ID NO: 34 - DNA, 51 bp, synthetic construct
  source 1..51: mol_type = other DNA
gctgttgaatattaatcgttaaatattcagatagacggagataataaacgg

SEQ ID NO: 35 - DNA, 29 bp, synthetic construct
  source 1..29: mol_type = other DNA
ggttgttgatcgaactcacgcccgacgcc

SEQ ID NO: 36 - DNA, 35 bp, synthetic construct
  source 1..35: mol_type = other DNA
ccagctcaccgtctgctttttgccgacaaagcgca

SEQ ID NO: 37 - DNA, 2302 bp, synthetic construct
  source 1..2302: mol_type = other DNA
caacagatcaaccggcaatttatccacggcatcaaattcgatcggttctttcaggcggacaaaaaagccggagacttttttcaggctgtcgagcttgccatgtggtatcgcaataccatgaccaaaacctgtcgaaccgagcgtttctctttcatgtaaccgttcggcgataaccttggggttaattagaaattctcgcccagcccattgggcaacctgctggaagagtgtttttttgttagaaggattgaaattcacctcaatcctgtccggcgcgattatatcggccagttcattcatgttttttcgcctttatatgcccacgattgcaaagatcgcagcatcagacattttctgaaagacacagcctttcagaaaataaagtcaaaattctagcaatattccttccggaaacgggggagaggggtcgataattctcttgaaaagagggagcgacctctctcccgtttattatctccgtctatctgaatatttaacgattaatattcaacagcaatagctggcttctcacctttggcgaaagcaactgctgcatcaaatgattggtgaaccatttctagaacagctttagttgtatagaaagccgtatgtggcgtaaccaatacattttcgcgtgaaatcaagtcagcaatcttagcatctgggaattctttaccagaccaatcttcattgaacaagccaacttcattttcataaacgtccataccgaagtctgaaatcttaccagaattcaaaccatcaataatagcatcaatgtccatcaaattaccacgcgcagcattcatgataaccacgccatccttcatcttagcaatagcctctgcattgattagatgatggttttcaggcacaccaggaacatacaatgaaattgcatcagcttgtgcatataattcgtctaatgtgtcaacgtacaaaccttctgcttgcaattcagcatttgggtacttatcataagcaataacctttgccccaaagcctttcaaaatgttaatagcaacacggccaatatggcctgtaccaataacaccaactgtttgcatacgcatttcacgtccaattgttggtgcccagcgcaagtcgtgcttagcaattttggcatccaatgctttcgtgcgacgtagcaaacgagataattgaatcattgagtgttctgcaatagcatttggtgaataaacaggaacatttgaaatgttaaagttaaattcacgtgctgcatcaaaatcaatgttatctgtaccaacgttacgcaatgacaagttagtaacaccaacgttagctaaagctgtcaatgtttcacgtgtatagtccagttgttggtaaacaacagctgaatctgatccctcagccaacttaactgtttcaggtgtcaatagctcttgtgtgtagtccacttcaatctctgggttagccgctttccattcttcaagtgatggcttttcatcatcacgaatgccgtaagcaaaaatcttcatgtttattctcctaacttattaagtagctactatattccatagctattttttaacgtgccgacttaccggcgatcgcggccaacaccttgttcgtgatgccgactgcggtcaagccgaaatgagcatatagatcattcgccggggccgatgcaccaaaaacatcaataccataacgaagaccatttattccagtataccgttcccagccaattgtcgtccctgcttcgatcgaaacgcgtaaaattgtcgattgaggctgatcgggcaaaacatcattacgataggattcgggttgttgatcgaactcacgcccgacgccagacgggattagaaattttgtcgataaacgcgccgtgacgcaataattcttgtgggctggcctcgtggggtcttggttgtcttttgacctttttattacgaggtagaccggaaaccgtgttcggggcagacaagcgtccatcggatgggccgttattgtcatccaaggccataccgatttgccgtccgcctgtcagttcaacataaagctgtgccagcaattcggcatcgagcaaggcgccatgtttgacgcgatggctgcggtcaataccgtagcgggtacataatgcgtcaagggtatgtttggcaccgggatgtttgccgcgcgcaatcgccaaggtatcgaccattcgggtcatacaaatggtcggaaggccgcatttatccaattcgtaattcaaaaagccgaaatcgaaggccgcattatgggcgaccaaaggcgacaatttgataaaggaaagcagttcttgcgctttgtcggcaaaaag

SEQ ID NO: 38 - RNA, 36 nt, synthetic construct
  source 1..36: mol_type = other RNA
gaaaagcccaagacttggcttgataatcttcaaaac

SEQ ID NO: 39 - DNA, 36 bp, synthetic construct
  source 1..36: mol_type = other DNA
gaaaagcccaagacttggcttgataatcttcaaaac

SEQ ID NO: 40 - DNA, 36 bp, synthetic construct
  source 1..36: mol_type = other DNA
gaacgttttgaagattatcaagccaagtcttgggct

SEQ ID NO: 41 - DNA, 39 bp, synthetic construct
  source 1..39: mol_type = other DNA
ccagctcaccgtctgccgtatccggtgtatcattttgag

SEQ ID NO: 42 - DNA, 43 bp, synthetic construct
  source 1..43: mol_type = other DNA
ggttgttgatcgaacgaacgaaaaaatttagctttagtgcgcg

SEQ ID NO: 43 - DNA, 36 bp, synthetic construct
  source 1..36: mol_type = other DNA
gctgttgaatattaaccaataggtcatctcttttgc

SEQ ID NO: 44 - DNA, 36 bp, synthetic construct
  source 1..36: mol_type = other DNA
agatctgatatcactacggggataaaaatcacttgc

SEQ ID NO: 45 - DNA, 3298 bp, synthetic construct
  source 1..3298: mol_type = other DNA
ccgtatccggtgtatcattttgaggtgcttctaaatgcacctgcgtaaaggcttgcgggtaatgttccttgataaggtgataagcgcgactataccattttgagccggggaagttggcgcctaaaaccgccgccgcatttcgggcttcggcacgaagccccaaagcaagatagctttccgttaaacgttccaaggcctctggcacgtaggttgttgtctgatattcgtcaataacacgccggaatcgtaaggtcgcggccaaccacagacggctttgttcataaaaacgaccgatcgccatttcttttccgcccagatggtcatgcaccaaatccatcttcaatcgggcatcggcggcataaggtgtatctggataccgccggataatgtcattcatggaatcaagagcaagctgggtggtgtgctgatcgtgattcacatcttgaatctgctcataataatccatcatgaccagatacatggcgtaaggtgcgtctttattacccgtatgaatggataaaaagcgctgtgccgaagcgatagaggctgaataattacgggcgcgataattacaaaaggcgctcatcaactgggcacggcgcgcccagatagaataaggatgctgtcgctcaacttcgtcaaagaaagccgcagcagccttatattgaccactgtccagactttgcttgccggcattataaagcgtatccacatcccgcgcgacataacgggtatcagtcttctttttaccccgaccggcacaaccagccatgggtaaaatcgcaatagcggtcagggctattaccaaggaacgagaaaattttgaggccatattctgcctctataatattttttcactcaaggaaacgggactctttcagtcacaggcctccttttaacagaaaaacagcgcttttgagctttcaaggaaacaactctaaaatttcatttctccaaacaaagccaattaaagcgcgcactaaagctaaattttttcgttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatatagtagctacttaataagttaggagaataaacatgaagatttttgcttacggcattcgtgatgatgaaaagccatcacttgaagaatggaaagcggctaacccagagattgaagtggactacacacaagagctattgacacctgaaacagttaagttggctgagggatcagattcagctgttgtttaccaacaactggactatacacgtgaaacattgacagctttagctaacgttggtgttactaacttgtcattgcgtaacgttggtacagataacattgattttgatgcagcacgtgaatttaactttaacatttcaaatgttcctgtttattcaccaaatgctattgcagaacactcaatgattcaattatctcgtttgctacgtcgcacgaaagcattggatgccaaaattgctaagcacgacttgcgctgggcaccaacaattggacgtgaaatgcgtatgcaaacagttggtgttattggtacaggccatattggccgtgttgctattaacattttgaaaggctttggggcaaaggttattgcttatgataagtacccaaatgctgaattgcaagcagaaggtttgtacgttgacacattagacgaattatatgcacaagctgatgcaatttcattgtatgttcctggtgtgcctgaaaaccatcatctaatcaatgcagaggctattgctaagatgaaggatggcgtggttatcatgaatgctgcgcgtggtaatttgatggacattgatgctattattgatggtttgaattctggtaagatttcagacttcggtatggacgtttatgaaaatgaagttggcttgttcaatgaagattggtctggtaaagaattcccagatgctaagattgctgacttgatttcacgcgaaaatgtattggttacgccacatacggctttctatacaactaaagctgttctagaaatggttcaccaatcatttgatgcagcagttgctttcgccaaaggtgagaagccagctattgctgttgaatattaaccaccaataggtcatctcttttgcccttaaaaatgatataaaaaagacgtcttctattctaatagaagacgcctctttctaaagagcaatcaatatcttttgattatttgctaaagctggtcggtgcagggtcaataacgacaataccattagctgatactttttcgacagctaacgcccgttcggcaatcgcctggttattaaagcggaaagcaccatcaatgcccataaagccattaggcgataacagcttcttttctggaaaatcgccattagaaggccaattcggagagatacgtgctgccagtaaaatagcatcatagccaaggctggctaagcgcggcggagtaacgccatagcgggctttatatttgccagataactggtggaacatcccatccggcacagaggcaaaccatgccccataaagggcgggaacatttttaagattattatcaatagaccaaagctcgctccccaatatctggggttttgtgccatcctgtttcacgagtaaaggcgctgcgatacgggcaattcgaggcgaatccgcaatcaaaacagcatcataatcctgtcccaatttggtcacggcgtcagtgatttcctgtggcgtgcggttataattggaaagggtcgcgacgctgcccccgttattggacaccgtgcgcagcaatgaagccgaggctttccggccataaaccccattgggaaggagggctgcaaagcgggataatttgtgatccaaagcatatttgacgacgcgctggataccagcacccaccgaaaagcccatcacataaacgccattgccagccaaactattgtcattggaaaaggtaatcatcgggatatgagcgttacgggcagcaagtgccatcattcgggcttcgctggggaaaaccccgccaaggattaacttattcccatcagaaacagctttgttgacagcctcggccgcgccttttgcactgtcatagcaagtgatttttatccccgt

SEQ ID NO: 46 - RNA, 32 nt, synthetic construct
  source 1..32: mol_type = other RNA
cacgaaagaacagaaatcgctggttaaaaatc

SEQ ID NO: 47 - DNA, 36 bp, synthetic construct
  source 1..36: mol_type = other DNA
gaaacacgaaagaacagaaatcgctggttaaaaatc

SEQ ID NO: 48 - DNA, 36 bp, synthetic construct
  source 1..36: mol_type = other DNA
gaacgatttttaaccagcgatttctgttctttcgtg

SEQ ID NO: 49 - DNA, 2782 bp, synthetic construct
  source 1..2782: mol_type = other DNA
ccgatccgccctatggtctgaccaatcttgatgaatccgacatcaaagagatgaactacaatggtgtgttccgtctctctcccgatggtcgtttagatttgatcgaagctggtcttagccgcccgaatggtttggctctgtccccggatgaaacgaaactttatgtttctaactcagaccgcgccagcccgaatatctgggtttattctttggatagcaatggcttgccaaccagccggactttgctgcggaacttccgcaaggaatatttcgatcagggtctcgccggtcttcctgatggtatgaatatcgacaaacagggcaatctttttgcttctgctccgggcggtatctatatttttgcaccagatggcgaatgcttgggtcttatttctggtaacccaggtcagccgttgtcgaactgctgctttggtgaaaaaggccagaccttgttcatctcggcttctcacaatgttgtgcgcgttcgcaccaaaacctttggataattattgtctgtcataaaaatataaaaaacagataatatacaaaattcaggcccgttttaagggaagtaatagaagaggatgccaaaaggcaaattcctgacgggccctgataagagaaagaacaaaagaattctttcttaagaatactctataatttggctatctttcttttctgaccgctttattgtcggaggatattccagagaagaaagtaagcaatcgttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatatagtagctacttaataagttaggagaataaacatgaagatttttgcttacggcattcgtgatgatgaaaagccatcacttgaagaatggaaagcggctaacccagagattgaagtggactacacacaagagctattgacacctgaaacagttaagttggctgagggatcagattcagctgttgtttaccaacaactggactatacacgtgaaacattgacagctttagctaacgttggtgttactaacttgtcattgcgtaacgttggtacagataacattgattttgatgcagcacgtgaatttaactttaacatttcaaatgttcctgtttattcaccaaatgctattgcagaacactcaatgattcaattatctcgtttgctacgtcgcacgaaagcattggatgccaaaattgctaagcacgacttgcgctgggcaccaacaattggacgtgaaatgcgtatgcaaacagttggtgttattggtacaggccatattggccgtgttgctattaacattttgaaaggctttggggcaaaggttattgcttatgataagtacccaaatgctgaattgcaagcagaaggtttgtacgttgacacattagacgaattatatgcacaagctgatgcaatttcattgtatgttcctggtgtgcctgaaaaccatcatctaatcaatgcagaggctattgctaagatgaaggatggcgtggttatcatgaatgctgcgcgtggtaatttgatggacattgatgctattattgatggtttgaattctggtaagatttcagacttcggtatggacgtttatgaaaatgaagttggcttgttcaatgaagattggtctggtaaagaattcccagatgctaagattgctgacttgatttcacgcgaaaatgtattggttacgccacatacggctttctatacaactaaagctgttctagaaatggttcaccaatcatttgatgcagcagttgctttcgccaaaggtgagaagccagctattgctgttgaatattaaaggtgcggtcttgattagccttgaagcctgacttcaaggctaagagaaaaaatagattttagataaagacaaccaaacatcctaagaataggatgttaatttataaccacgccgtgtttttttgcccataattcaagctggtcaccaatattgctggcaccgcttgcaaccaattcccgaataaagggcgcaaaagcatcccgatccaaagagcggatcattgccttgataggccctatcgccactggtgtgatagaaagccgttttaagcccaatccgatgagtgccatggccgtcagagtacgaccacccatttccccgcaaatgccgaccttacaacccgcagcattggcctgatcgataatccgtgacaagaaacgcagcaaagccggagagagccagtcatatctttctgccagcttgggataagcacgatcggctgcaaaaaagaactggatcagatcattagtgccaatcgacaagaaatcgagcttgggcaacagaatatcaagcgtctcggctaaggaaggcacctctagcatcgagccataataaattctcttcgcgcgcggtttgtcatgtttggcaagccaagcattttgcttttcaaagacagcgcgagcctgttcatattcccatggctcggcaatcatcgggaacatgacataaagcgtttttcccgcagccccttcaatcaatgcccgtgcctgaaccttgagcagggctttcttttcgagagccaagcggatagcac